(19) United States
(12) Patent Application Publication
(10) Pub. No.: US 2003/0206982 A1
(43) Pub. Date: Nov. 6, 2003

(54) Method of improving dietary balance using gustatory and olfactory aliesthesia

(76) Inventors: Antje Spors, Los Angeles, CA (US); Laurent Bronner, Los Angeles, CA (US)

Correspondence Address: THOMAS S. KEATY, KEATY PROFESSIONAL LAW CORP., 2140 WORLD TRADE CENTER, NO. 2 CANAL STREET, NEW ORLEANS, LA, 70130, US

(21) Appl. No.: 10/410,767

(22) Filed: Apr. 10, 2003

Related U.S. Application Data
(63) Continuation-in-part of application No. 10/131,765, filed on Apr. 24, 2002, now abandoned.

(51) Int. Cl. A61K 35/78; A61K 38/00

(52) U.S. Cl. 424/777; 424/725; 514/2

(57) ABSTRACT

A method of inducing weight loss and promoting homeostasis in an individual relies on olfactory and gustatory reaction of the individual to a wide variety of offered groups of food items. The individual is encouraged to select one particular type of a food item, for instance one type of fruit, based on positive alliesthesial response, and consume this type of food item until negative alliesthesia is detected. Once alliesthesia is reached, the individual is encouraged to stop eating. The next meal, removed by several hours from the first meal, relies on similar concepts.

### CROSS-REFERENCE TO RELATED APPLICATIONS

This application is a continuation-in-part of our co-pending application Ser. No. 10/131,765 filed on Apr. 24, 2002 entitled “A Method of Inducing Weight Loss and improving Dietary Balance Using Olfactory and Gustatory Alliesthesia,” the full disclosure of which is incorporated by reference herein.

### BACKGROUND OF THE INVENITON

This invention relates generally to a method of regulating dietary balance, inducing weight loss, selecting food, regulating the quantity of the food-intake and to a method of altering habitual eating patterns in individuals desiring to decrease weight through a dietary balance. It is well known that unhealthy eating habits -and dietary imbalance bring about weight problems, deterioration in individual's health, increased susceptibility to diseases and deterioration in the quality of life.

Paleontologists and anthropologists believe that in the past, Man was not suffering from diseases of civilization, such as heart diseases, cancer, diabetes, etc., and that Man was certainly not overweight. For most part of their evolutionary history, humans, like all other inhabitants of this planet were exposed only to natural, unprocessed food items, the way they were available in the wild. Food gathering brought raw eatable plant parts, eggs and honey, hunting brought wild game, fishing brought seafood.

Since the beginning of life, species have managed to achieve dietary balance despite the absence of dietetic knowledge. For all species, evolution has perfected sensory mechanisms allowing appropriate food selection and food-intake regulation to ensure optimal health and survival. Only individuals with the sensory mechanisms working at their best survived and passed their genes to the next generation, constantly improving the ability to select appropriate food items and regulate the intake quantity-according the nutritional needs. In early stages of human evolution, Man, like any other mammal still today, relied only on olfactory receptors for accurate food selection and gustatory receptors for precise food-intake quantity regulation.

During pre-paleolithic period (6 million-2.5 million years ago) our ancestors did not posses tools for cutting meat, they did not use fire in food preparation and had to consume all their foods in an unaltered state.

As technology progressed, natural foods were altered. Food has been processed in a variety of ways, from heating to freezing, from mechanical to chemical processing, from artificial selection to genetic engineering, etc. Any food processing technique creates new chemical compounds that did not exist in a pre-Paleolithic era. Because of the fast and relatively recent introduction of constantly changing food processing techniques, and the significant amount of time (millions of years) the human genetic makeup needs to adapt to new chemical compounds, the sensory system is unable to decode the new chemical information correctly. Therefore, food intake regulation and food selection can no longer be accurately determined by olfactory and gustatory receptors.

Technology developed numerous ways to “enhance” the taste and flavor of natural unprocessed food items. Enhanced tastes tempt people to eat processed food beyond the actual nutritional needs of the body. The food industry continues to provide taste enhancers and modifiers, which tend to further alter taste and smell. As a result, eating preferences and patterns have drastically changed. The end products of food processing and food preparation, misleading the chemical senses, became abnormally attractive, and unchecked consumption of processed food became the culprit of many diseases.

The resultant absence of sensory guidance gave rise to the diet industry and nutritional sciences, both of which try to conquer the effects of lifelong excessive and deficient eating habits, and suggest different approaches to improve dietary balance. In all instances, diets follow a theory-diagnosis-prescription procedure and consist of “daily average requirements” of nutrients to be consumed for dietary balance in the individual.

Many diets rely on a certain number of calories to be consumed during the day regardless of what type of food a person eats. Other diets stress the importance of relying on the USDA's food pyramid. Many are based on nutritional supplements to compensate for the fact that food processing, such as cooking, destroys nutritional elements (vitamins, enzymes, etc.) eventually leading to deficiencies. Still others rely on medications to control and curb appetite.

However, diets and the concept of daily average requirements fail to take into consideration the biochemical state of the body at a given time. In addition, the needs of one individual may significantly differ from the needs of another person or statistical averages. Those nutritional needs equally change significantly during the day as well as from day to day.

Depending on the type of activity (mental, physical, rest, etc.) and depending on extraordinary circumstances (pregnancy, menstruation, growth, disease, injury, etc), the body is in need of different nutrients at different times. For example, at one particular day an individual may be engaged in intensive mental activities and needs higher doses of phosphor than usual, while on other days, the same individual may be involved in physical activities and need higher than usual doses of energy carbohydrates. Under such circumstances, the usual theory-diagnosis-prescription procedure cannot follow the real-time needs of the body. Attempts to monitor the body's nutritional needs have been made with techniques such as blood sampling, hair analysis, urine analysis, etc. involving highly sophisticated laboratory equipment, extensive costs and time consuming procedures. Even in the case where repeated diagnosis could be achieved, the complexity of the biochemical processes involved in nutrition is a major obstacle to evaluation of the entire spectrum of nutrients the body might need. As a result, diagnosis may be incomplete, making the prescription inaccurate and sometimes even unsafe.

With the progress of civilization, as more flavor-enhanced foods become available, the olfactory and gustatory receptors can no longer be relied upon for selecting the appropriate food and controlling the food intake quantity. However, it is believed that particular nutritional instincts can be relied upon when allowing an individual -to choose a particular food from a selection of strictly unprocessed food items. The “unprocessed food” is defined here as food that had not been exposed to temperatures below freezing (32 degrees Fahrenheit) or heated over 104 degrees Fahrenheit at any time. It also means that the food was not exposed to mechanical denaturing through smashing, grinding, slicing, juicing, mixing, etc.; that no seasoning was added. Unprocessed food also includes products that have not been exposed to chemical fertilizers, heated fertilizers, have not been genetically modified, over hybridized or artificially over selected, such as vegetal products, wheat, corn and soy. Unprocessed food will exclude selectively bred animals (beef, sheep, etc.) and meat from animals or fish that have not been left in their natural state and environment during the last two generations. Unprocessed foods here also mean that no animal milk, dairy products or by-products are used.

One of the approaches in regulating dietary balance was developed by Canadian scientist Dr. Michel Cabanac who researches physiology of behavior associated with food intake activity; his research ranges from comparative physiology of temperature regulation (especially invertebrates) to human temperature regulation and to the regulation of body weight. Dr. Cabanac coined a term “alliesthesia.” In its basic definition, alliesthesia refers to perception of the same external stimulus as sometimes pleasant and sometimes unpleasant according to the state of the milieu interieur. The seeking of pleasure and the avoidance of displeasure lead to behaviors with useful homeostatic consequences.

However, Dr. Cabanac's method does not differentiate between processed and unprocessed foods; it does not provide for the use of a wide variety of food products made available to an individual, whose alliesthesial response is evaluated. In one of Dr. Cabanac's papers, he also suggests using “unpalatable” food to promote weight loss and discourage over-eating. Putting the alliesthesial phenomenon in a context of a wide array of unprocessed food items—the context in which the phenomenon has evolved, and the exclusive consumption of highly pleasurable food items are the novelties that set the method of the present invention apart from Cabanac's research.

The present invention contemplates provision of a method of achieving dietary balance and encouraging weight loss though the use of olfactory and gustatory responses of an individual at a particular meal.

### SUMMARY OF THE INVENTION

It is, therefore, an object of the present invention to provide a method of inducing weight loss in individuals and improving dietary balance through careful selection of food items for immediate consumption.

It is another object of the present invention to provide a system that uses individual's olfactory alliesthesia in food selection as indicator for the individual's needs.

It is a further object of the present invention to provide a system that uses individual's gustatory alliesthesia in food intake regulation as indicator for the individual's needs.

It is still a further object of the present invention to provide a method inducing behavioral modifications in individuals to induce weight loss and improve dietary balance.

These and other objects of the invention are achieved through a provision of a method of inducing weight loss, improving dietary balance and promoting homeostasis, which relies on olfactory and gustatory alliesthesial response of the individual to a vide variety of different groups of food items. The groups of food items available for selection include a variety of strictly unprocessed foods: fruits, dates and honey (Group I), vegetables and herbs (Group II, and fat- and protein-containing foods (Group III).

The individual is encouraged to select one particular type of a food item using positive olfactory alliesthesia, for instance one type of fruit. The individual is encouraged to consume this type of food item until negative gustatory alliesthesia or another alliesthesial response (for instance repletion) is achieved. At this point, the individual is encouraged to stop eating this particular type of food. The steps above can be repeated within the same meal until satiation is reached.

The next meal, removed by at least 4 to 5 hours from the first meal relies on similar steps. By encouraging the individual to respond to the individual's instinctive preferences for these specific items, the individual's weight loss and dietary balance are induced.

### DETAILED DESCRIPTION OF THE PREFERRED EMBODIMENT

The method of the present invention relies on individual's alliesthesial response to particular food groups and food species during meals. The method of the present invention further stresses the need for consumption of foods in their natural state that is unprocessed, raw foods, as the term is defined above. The present method utilizes what is called here “pre-Paleolithic” diet in combination with sensory-regulated alliesthesial response of an individual.

Studies demonstrate that pleasure aroused by eating shows an identical pattern. A given alimentary flavor is described as pleasant during hunger and becomes unpleasant or indifferent during satiety. Measurement of human ingestive behavior confirms the above relationship of behavior with pleasure. It has been repeatedly demonstrated in the case of food intake that human subjects tend to consume foods that they report to be pleasant and to avoid those that they report to be unpleasant. Pleasure also shows a quantitative influence: the amount of pleasurable food eaten is a function of alimentary restrictions, and increases after dieting. The result is that pleasure scales can be used to judge the acceptability of food.

The seeking of pleasure and the avoidance of displeasure lead to behaviors with useful homeostatic consequences; it motivates the subject to select most pleasant food items and stop food consumption as soon as the unpleasant sensation, or negative alliesthesia is detected. According to this method, the physiological phenomena wherby internal signals of an organism influence the perceived pleasant or unpleasant interpretation of external stimuli, i.e. gustatory and olfactory stimuli are used to modify and regulate behavior of a subject.

It may be difficult to apply the principle of alliesthesial response to processed foods. Table 1 compares gustatory alliesthesial response between non-denatured (unprocessed) and processed foods.

| | | | Gustatory |
|---|---|---|---|
| Non- | Gustatory Alliesthsial | | Alliesthesial Response |
| denatured | Response when Dietary | Processed | when Dietary Needs |
| food | Needs are Met | Food | are Met |
| Leek | Very accurate | Steam- | Weak to nonexistent |
| | Biting Taste | ed leek | |
| Carrot | Very accurate | Cooked | Weak to nonexistent |
| | SoaDv taste | Carrots | |
| Raw | Very accurate | Heated | Weak to nonexistent |
| Honey | Burning throat | honey | |
| Cacao | Very accurate | Choco- | Weak to nonexistent |
| bean | Bitter teaste | late | |
| Straw- | Very accurate | Straw- | Weak to nonexistent |
| berry | Acidic taste | berry | |
| | | ice | |
| | | cream | |
| Raw | Very accurate | French | Weak to nonexistent |
| Potato | Biting taste | fries | |
| Rasberry | Very accurate | Ras- | Weak to nonexistent |
| | Acidic taste | berry | |
| | | Jam | |

One can easily overeat on processed foods because they have been chemically transformed in a way that weakens the body's alliesthesial signals so that everyone can eat them even if there is no dietary need. With processed foods and recipes, alliesthesia cannot lead to the ideal weight and promote homeostasis because of the nature of culinary art, which is to make food palatable at any time for any individual. With chemically nonaltered foods, on the other hand, gustatory and olfactory alliesthesia has been and still is the major guide for animals to ensure well-being and dietary balance. The same is true for humans as long as they find themselves in the appropriate food context. The method of the present invention puts the alliesthesial function in precise context of weight loss and body balance.

Based on the widely recognized principles of homeostasis, a living organism, such as human body, seeks to maintain an equilibrium, or optimal conditions for survival, by means of automatic mechanisms that counteract influences tending toward disequilibrium. The application of the concept of homeostasis to the quantity and variety of food required by a human body for maintenance under different external conditions serves to support the principle of negative feedback as is achieved when a certain nutrient is missing or in over-abundance in a human body. The self-regulating mechanism in a living system of a human body involves constant monitoring and regulating of numerous factors, including the gases oxygen and carbon dioxide, nutrients, toxins, hormones, and organic and inorganic substances. For a human body to be considered healthy the concentrations of these substances in body fluid must remain within genetically determined limits, despite adverse changes in the internal and external environment.

The internal equilibrium of the body, the ultimate gauge of its proper functioning, involves the maintenance of a constant rate of concentration in the blood of certain molecules and ions that are essential to life and the maintenance at specified levels of other physical parameters such as body temperature. Similarly, homeostasis regulates the stability of our body fluids to sustain life; these include properties like temperature, salinity, acidity, and the concentrations of nutrients and the detoxification of toxins and dietary antigen. Because these properties affect the chemical reactions that keep us alive, we have built-in alliesthesial and physiological mechanisms to maintain them at desirable levels.

Based on the above concepts, a study was conducted to evaluate the effectiveness of the method of weight loss and improvement of dietary balance using sensory-regulated pre-Paleolthic diet. The objectives were to measure the weight loss induced by the method of the present invention within a 21-day period; measure the improvements in dietary balance and measure the level of satisfaction and eating pleasure that can be achieved with the exclusive consumption of strictly unprocessed foods selected on the basis of olfactory alliesthesia alone.

Olfactory receptors usually respond to the smell of certain substances. A subject is encouraged to select a food substance from a pre-determined group of food substances. Food groups are chosen from fruits and sugar-containing foods, such as dates and honey (Group I), vegetables and herbs (Group II) and protein/fatty-rich foods (Group III).

All individuals were required to report to the test center for 2 meals each day. Eight women and two men (study participants) were presented at each meal with the broadest possible array of strictly unprocessed food substances. All subjects were asked to select food items based on olfactory alliesthesia. Only the items with the most pleasant odors were consumed. In some cases, blindfold was used to avoid visual influences. No prescriptions, limitations or restrictions were applied when food selection was made. The quantity of consumed food was regulated by the subject's own alliesthesial signals.

During tests, negative alliesthesia and sensory-specific satiety were used. Consumption of a food substance from a particular food group was encouraged until negative gustatory alliesthesia or satiety was reached. It is believed that stimulation of the intestinal tract that signals the brain to indicate that a particular nutrient has been sufficiently consumed plays a role in negative gustatory alliesthesia. Another criteria that may be used in evaluation of the sufficient food intake is satiation, the process that leads to an eating occasion being brought to an end. It implies an evaluation of the foods which anticipates the effects linked to their digestion and their metabolic use. These mechanisms act together to monitor food intake.

It is believed that one component of the perception of foods, namely pleasure/displeasure, is modified during satiation by separate mechanisms which have the effect of putting a stop to eating by reducing the pleasure perceived during ingestion of the foods. One of these mechanisms is negative alliesthesia mentioned above.

Another mechanism is often referred to as sensory-specific satiety. This mechanism is triggered when the same taste, texture or flavor is used continuously to quickly discourage consumption of that particular food substance. The sensory-specific satiety is an important mechanism for encouraging people to eat a variety of foods and therefore obtain the necessary variety of nutrients.

The consumed foods were unprocessed. All foods were consumed separately without mixing, seasoning, or other processing or preparation technique. The following food selections were made available for lunch meal (12-2 pm):

Group I: Fruits: apples, apple bananas, bananas, cherimoyas, clementines, guavas, grapefruits, jackfruit, chempedack, kiwis, kumquats, lemons, mandarins, oranges, papayas, passion fruit, maracuja, pears, persimmons, pineapples, pineapple guavas, pomelo, sapote blanco, sapote mamey, strawberries, tangelos.

Dates: 9 different date varieties (black seedlings, black precioso, honey, etc)

Food choices for dinner meal (6-8pm):

Group III: Protein/fatty-rich foods: avocados, carob, buffalo meat, coconuts, deer meat, macadamia nuts, mackerel, muscles, pecan nuts, pistachios, walnuts, rock crabs, sardines, shrimps, spider crabs, squid, sword fish, tuna, yellow tail, snap peas.

Group II: Vegetables: artichokes, asparagus, broccoli, cabbage, carrots, cauliflower, celery, Chinese cabbage, garlic, leek, lettuce, mint, onion, parsley, parsnip, radish, spinach, squash, turnip, yam, and many others. This Group also includes a variety of herbs.

Group I: Fruits (if no animal foods have been consumed) fruits: apples, apple bananas, bananas, cherimoyas, clementines, guavas, grapefruits, jackfruit, chempedack, kiwis, kumquats, lemons, mandarins, maracuja, oranges, papayas, passion fruit, pears, persimmons, pineapples, pineapple guavas, pomelo, sapote blanco, sapote mamey, strawberries, tangelos.

Dates: 9 different date varieties (black seedlings, black precioso, honey, etc)

All subjects have been encouraged to drink spring water as only beverage in between and before meals. No water was consumed during or within 30 min after the meals. Unprocessed herbs were used also in between meals to control detoxification symptoms.

In order to allow for self-regulation of the intake quantity, subjects were asked to stop the consumption of each food at the first signs of gustatory alliesthesia or other similar internal bodily signal. Once satiety was achieved, individuals were asked to stop the food consumption. The participants were asked to consume only what taste and smelled the most delicious within the available food choices until they felt satisfied.

Tables 2 and 3 below present Summary of the results based on body weight loss and body mass index.

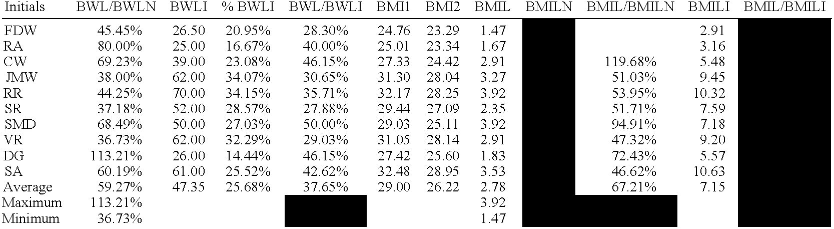

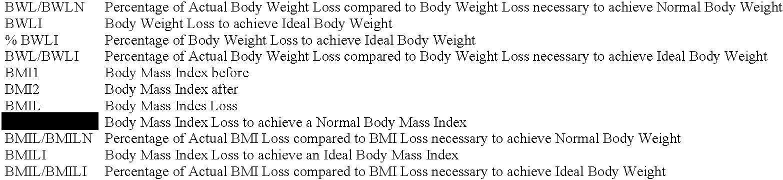

B) Eating Pleasure and Satisfaction Levels

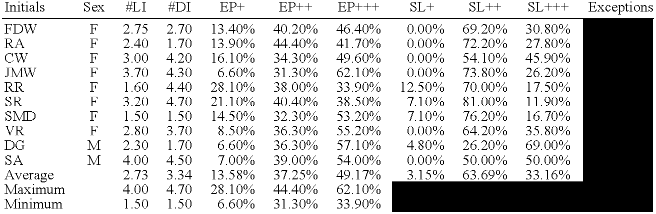

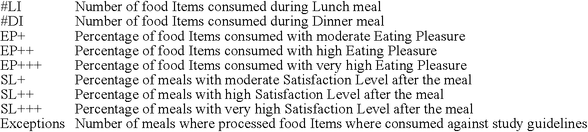

The studies provided the following results:

Average of actual body weight loss (BWL) within the 21-day period: 17.5 lbs

Maximum BWL in one subject within the 21-day period: 26 lbs

Minimum BWL in one subject within the 21-day period: 7.5 lbs

Average BWL needed to reach normal weight would have been: 33.98 lbs

Average percentage of actual BWL in relation to total body weight: 9.43%

Average percentage of BWL needed to reach normal weight would have been: 18.24%

The average of actual BWL in relation to BWL needed to reach normal weight is: 59.27%

Average BWL needed to reach ideal weight would have been: 47.35 lbs

Average of actual BWL Percentage in relation to total body weight: 9.43%

Average BWL Percentage needed to reach ideal weight would have been: 25.68%

The average of actual BWL in relation to BWL needed to reach ideal weight is: 36.72%

Average actual Body Mass Index (BMI) Loss is: 2.78

Average BMI Loss needed not to be considered overweight anymore would have been: 5.13

Percentage of actual BMI Loss in relation to normal BMI Loss: 67.21%

BMI Loss needed to reach ideal BMI would have been: 7.15

Percentage of actual BMI Loss in relation to BMI Loss needed to reach ideal BMI: 41.21%

Within a group of 10 subjects and the application of a sensory-regulated pre-Paleolithic diet during a 21-day period, subjects have lost an average of 17.5 lbs, which represents an average of 59% of the weight they had to loose so not to be considered overweight anymore. They also lost an average of 37% of the weight they needed to loose to reach their ideal weight.

Furthermore, individuals dropped their BMI by an average of 2.78. This represents a 67% fraction of the BMI they had to lose so not to be considered over weight anymore and a 41% fraction from the total Bmlloss needed to reach their ideal BMI. The results of the study were also evaluated based on meal satisfaction and eating pleasure.

As illustrated in Table 4, in average, subjects have consumed 2.7 food items for lunch and 3.3 food items for dinner to be satisfied. Overall, subjects described their level of satisfaction at the end of the meals as moderate: 3.2%, high: 63.7% and very high: 30.6%.

The overall level of eating pleasure the subjects have described with the different food items consumed ranges from 13.6% for moderate levels of eating pleasure, 37.3% of high levels of eating pleasure and 49.2% very high levels of eating pleasure.

The results of the study were further evaluated based on blood work of the participants. Tables 5-9 illustrate these results.

C) Blood Work

| Initials | Sex | Age | BH (feet) | BP 1 | BP 2 | Pulse 1 | Pulse 2 | PH 1 | PH 2 | Glucosis 1 | Glucosis 2 | Gl. Ch. |
|---|---|---|---|---|---|---|---|---|---|---|---|---|
| FDW | F | 57 | 5′ | 100/70 | 100/70 | 80 | 80 | 7 | 6.5 | | | |
| RA | F | 60 | 5′5″ | 110/84 | 100/82 | 60 | 64 | 6 | 6.5 | | | |
| CW | F | 35 | 5′6″ | | | | | | | | | |
| JMW | F | 47 | 5′4″ | | | | | | | | | |
| RR | F | 55 | 5′7″ | 130/90 | 114/80 | 76 | 84 | 6.3 | 7 | | | |
| SR | F | 47 | 5′6″ | 110/80 | 100/78 | 64 | 68 | 6.5 | 6 | | | |
| SMD | F | 27 | 5′7″ | | | | | | | | | |
| VR | F | 34 | 5′6″ | | | | | | | | | |
| DG | M | 58 | 5′8″ | | | | | | | | | |
| SA | M | 47 | 6′ | 150/90 | 140/90 | 60 | 72 | 5.5 | 6 | 248 | 135 | 45.56% |
| # of O.R.V | | | | | | | | | | 1 | 1 | |
| A. Ch. | | | | | | | | | | | | 45.56% |

▪ Out of Range Values or percentages of change transforming an in range value into an out of range value
# of O.R.V # of Out of Range Values
A. Ch. Average Change; positive percentages are the percentages of decreasing values, negative percentages are the ones of increasing values

| Initials | BUN 1 | BUN 2 | BUN Ch. | BUN Creat. 1 | BUN Creat. 2 | BUN Creat. Ch. | Calicium 1 | Calcicium 2 | Calcium Ch. |
|---|---|---|---|---|---|---|---|---|---|
| FDW | | | | | | | | | |
| RA | | | | 12 | 5.5 | 54.17% | 10.6 | 10.1 | 4.72% |
| CW | | | | | | | | | |
| JMW | | | | | | | | | |
| RR | | | | | | | | | |
| SR | | | | | | | | | |
| SMD | | | | | | | | | |
| VR | | | | | | | | | |
| DG | | | | | | | | | |
| SA | 22 | 26 | −18.18% | | | | | | |
| # of O.R.V | | 1 | | | 1 | | 1 | | |
| A. Ch. | | | −18.18% | | | 54.17% | | | 4.72% |

▪ Out of Range Values or percentages of change transforming an in range value into an out of range value
# of O.R.V # of Out of Range Values
A. Ch. Average Change; positive percentages are the percentages of decreasing values, negative percentages are the ones of increasing values
BUN Creat. BUN Creatinine

| Initials | Cholesterol 1 | Cholesterol 2 | Chol. Ch. | Trigl. 1 | Trigl. 2 | Trigl. Ch. | HDL 1 | HDL 2 | HDL Ch. |
|---|---|---|---|---|---|---|---|---|---|
| FDW | 235 | 197 | 16.17% | | | | | | |
| RA | 291 | 181 | 37.80% | | | | | | |
| CW | | | | | | | | | |
| JMW | | | | | | | | | |
| RR | | | | | | | | | |
| SR | 212 | 159 | 25.00% | 180 | 74 | 58.89% | | | |
| SMD | | | | | | | | | |
| VR | | | | | | | | | |
| DG | | | | | | | | | |
| SA | 209 | 192 | 8.13% | 205 | 118 | 42.44% | 32 | 44 | −37.50% |
| O.R.V | 4 | | | 2 | | | 1 | | |
| A. Ch. | | | 21.78% | | | 50.66% | | | −37.50% |

▪ Out of Range Values or percentages of change transforming an in range value into an out of range value
# of O.R.V # of Out of Range Values
A. Ch. Average Change; positive percentages are the percentages of decreasing values, negative percentages are the ones of increasing values
Trigl. 1 Triglycerides

| Initials | LDL 1 | LDL 2 | LDL Ch. | CHOL/HDL R. R. 1 | CHOL/HDL R. R. 2 | CHOL/HDL R. R. Ch. | ROW 1 | ROW 2 | ROW Ch. |
|---|---|---|---|---|---|---|---|---|---|
| FDW | 145 | 126 | 13.10% | | | | 10.6 | 10.8 | −1.89% |
| RA | 216 | 116 | 46.30% | 5.4 | 3.7 | 31.48% | | | |
| CW | | | | | | | | | |
| JMW | | | | | | | | | |
| RR | | | | | | | | | |
| SR | | | | | | | | | |
| SMD | | | | | | | | | |
| VR | | | | | | | | | |
| DG | | | | | | | | | |
| SA | 136 | 124 | 8.82% | 6.5 | 4.4 | 32.31% | | | |
| O.R.V | 3 | | | 2 | | | 1 | | |
| A. Ch. | | | 22.74% | | | 31.89% | | | −1.89% |

▪ Out of Range Values or percentages of change transforming an in range value into an out of range value
# of O.R.V # of Out of Range Values
A. Ch. Average Change; positive percentages are the percentages of decreasing values, negative percentages are the ones of increasing values
CHOL/HDL R. R. 1 CHOL/HDL RISK RATIO

| | T4 | T4 | T4 | MPV | MPV | |
|---|---|---|---|---|---|---|
| Initials | Thyro. 1 | Thyro. 2 | Thyro. Ch. | 1 | 2 | MPV Ch. |
| FDW | | | | | | |
| RA | | | | | | |
| CW | | | | | | |
| JMW | | | | | | |
| RR | | | | | | |
| SR | 4.3 | 5.8 | −34.88% | 6.9 | 8.4 | −21.74% |
| SMD | | | | | | |
| VR | | | | | | |
| DG | | | | | | |
| SA | | | | | | |
| O.R.V. | 1 | | | 1 | | |
| A. Ch. | | | −34.88% | | | −21.74% |

▪ Out of Range Values or percentages of change transforming an in range value into an out of range value
# of O.R.V. # of Out of Range Values
A. Ch. Average Change; positive percentages are the percentages of decreasing values, negative percentages are the ones of increasing values
T4 Thyro. T4 Thyroxine

### SUMMERY

Page 8

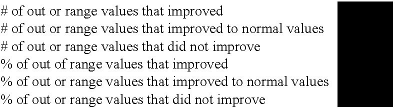

All subjects, who had their cholesterol values out of range, normalized their values within a 21-day application of a sensory-regulated pre-Paleolithic diet.

All subjects, who had their triglycerides values out of range, normalized their values within a 21-day application of a sensory-regulated pre-Paleolithic diet.

All subjects, who had their LDH cholesterol values out of range, normalized their values within a 21-day application of a sensory-regulated pre-Paleolthic diet.

All subjects, who had their cholesterol/HDL risk ratio out of range, normalized their values within a 21-day application of a sensory-regulated pre-Paleolithic diet.

In one subject, who had been diagnosed with diabetes, the glucose level in the blood work dropped from the out of range value 248 to the normalized value 135 within a 21-day application of a sensory-regulated pre-Paleolithic diet, even tough the subject stopped taking insulin and any other diabetes related medication a few days after the beginning of the study and despite of a large consumption of fruits and dates.

Overall, 89.5% of values out of range improved, 84.2% of values out of range went back to normal, 10.5% did not improve.

During the study, the following parameters were taken before and after the 21-day period for all participants (parameters measured before the 21-day period are indicated as “1)”, after the 21-day period as “2)”:

Body Weight (BW)Additional Parameters are taken on most of the individuals:

Blood Pressure (BP)

Blood Work: SMA25 (chart contains only out of range measures)

The following parameters have been calculated based on indicated references:

Ideal Body Weight (IBW); Ref.: Calorie Control Council

Normal Body Weight allows a 10% range compared to ideal Body Weight; Ref.:

Calorie Control Council (NBW=(IBW)*1.1)

Actual Body Weight Loss (BWL=BW1-BW2)

Body Weight Loss to reach Normal Body Weight (BWLN=BW1-NBW)

Percentage of Body Weight Loss compared to original weight (% BWL=[BWL/BW 1]*100)

Percentage of Body Weight Loss to reach Normal Body Weight compared to original weight (% BWL=[BWLN/BW1]*100)

Percentage of Actual Body Weight Loss compared to Body Weight Loss to reach Normal Weight (% BWUBWLN=[BWUBWLN]*1 00)

Ideal Body Weight Loss is (BWLI=BW1-IBW)

Percentage of Ideal Body Weight Loss compared to reach Ideal Body Weight (%BWL =[BWLI/BW1]*1 00)

Percentage of Actual BWL compared to Body Weight Loss to reach Ideal Body Weight (%BWUBWLI=[BWUBWLI]*1 00)

Actual Body Mass Index (BMI=[BW*704.5]/[BH*BH])

Actual Body Mass Index Loss (BMIL=BMI1-BMI2)

Normal Body Mass Index is between 19 and 24.9. A person with a BMI over 25 is considered overweight.

Ideal Body Mass Index is set at 21.85

Body Mass Index Loss to reach a Normal Body Mass Index (BMILN=BMI1-24.9)

Percentage of Actual Body Mass Index Loss compared to Normal Body Mass Index Loss (%BMIL/BMILN=[BMIL/BMILN]*100

Body Mass Index Loss to reach an Ideal Body Mass Index (BMILI=BMI1-21.85)

Percentage of Actual Body Mass Index Loss compared to Ideal Body Mass Index Loss (%BMIL/BMILI=[BMIUBMILI]*1 00

Average number of items consumed in lunch meals (#LI)

Average number of items consumed in dinner meals (#DI)

Average percentage of food items consumed with moderate eating pleasure (EP+)

Average percentage of food items consumed with high eating pleasure (EP++)

Average percentage of food items consumed with very high eating pleasure (EP+++)

Average percentage of moderate satisfaction level after meals (SL+)

Average percentage of high satisfaction level after meals (SL++)

Average percentage of very high satisfaction level after meals (SL+++)

According to the method of the present invention, the food sources include concentrated sugars, such as dates and honey, and highly fatty and high protein foods, such as avocados, macadamia nuts, tuna fish and pistachios. Such foods are commonly known to generate weight gain, but in the study, these foods were consumed in a significant amount and the individuals still lost weight. It is believed that alliesthesial guidance and the absence of processed foods are to a large part responsible for the beneficial outcome. It is also noted that perhaps milk and dairy products due to their recent introduction in evolutionary history do not stimulate the necessary alliesthesial response to properly regulate the amount of the consumed product. Hence, these products were excluded from the study.

The individuals in the study were encouraged to select one species of food items from a wide variety of food items within a particular group. From the provided source, the individuals were requested to select a particular food item with the highest olfactory alliesthesial response and to continue consuming that type of item until negative gustatory alliesthesia, or repletion, or satiety was detected. The individuals were then requested not to return to the same type of the food items during the same meal.

Many changes and modifications may be made in the method of the present invention without departing from the spirit thereof We therefore pray that our rights to the present invention be limited only by the scope of the appended claims.

We claim:

1. A method of inducing weight loss and improving dietary balance in individuals, comprising the following steps:
Providing a source of different groups of unprocessed food items;
selecting from the provided source one species of food items based on positive olfactory alliesthesia of an individual; and
consuming the selected species of food items until negative gustatory alliesthesia is achieved.

2. The method of claim 1, wherein the selected species of food items facilitates highest positive olfactory response and highest positive gustatory response.

3. The method of claim 1, wherein any selected food item can only be consumed once during the same meal.

4. The method of claim 1, wherein the unprocessed food items comprise food items that have not undergone thermal treatment below 32 degrees and above 104 degrees Fahrenheit.

5. The method of claim 1, wherein at least one group of food items comprises fruits.

6. The method of claim 1, wherein at least one group of food items comprises vegetables.

7. The method of claim 1, wherein at least one group of food items comprises protein- and fat-containing food items.

8. The method of claim 1, wherein at least one group of food items comprises herbs.

9. The method of claim 1, wherein at least one group of food items comprises dates and honey.

10. The method of claim 8, wherein the group of protein- and fat-containing food items comprises animal-based proteins and fats.

11. The method of claim 11, wherein the source of food items for animal-based protein and fats comprises selection of animals that are free from selective breeding and are the product of at least two-generation feeding as found in a natural habitat.

12. The method of claim 11, wherein the group of animal-based proteins and fats excludes milk and dairy products.

13. The method of claim 1, wherein two meals are consumed during a day, with each food group being presented at least once a day.

14. A method of inducing weight loss and facilitating homeostasis in an individual, comprising the following steps:
Providing a source of different groups of unprocessed fruits, dates and honey, vegetables, herbs, protein- and fat-containing foods, selecting from the provided source a desirable species of food items based on positive olfactory alliesthesia of the individual, and consuming the selected species of food items until achieving negative alliesthesia or repletion.

15. The method of claim 15, wherein the unprocessed foods comprise food items that have not undergone thermal treatment below 32 degrees and above 104 degrees Fahrenheit.

16. The method of claim 15, wherein said group of protein- and fat-containing foods comprises animal-based proteins and fats.
